(19) United States
(12) Patent Application Publication
(10) Pub. No.: US 2008/0227814 A1
(43) Pub. Date: Sep. 18, 2008

(54) Selective Estrogen Receptor Modulators

(76) Inventors: Jeffrey Alan Dodge, Indianapolis, IN (US); Scott Alan Frank, Indianapolis, IN (US); Ronald Jay Hinklin, Longmont, CO (US); Owen Brendan Wallace, Zionsville, IN (US)

Correspondence Address: ELI LILLY & COMPANY, PATENT DIVISION, P.O. BOX 6288, INDIANAPOLIS, IN, 46206-6288, US

(21) Appl. No.: 10/597,165

(22) PCT Filed: Jan. 18, 2005
(86) PCT No.: PCT/US05/00018

§ 371 Date: Jul. 13, 2006

Related U.S. Application Data
(60) Provisional application No. 60/539,938, filed on Jan. 29, 2004.

(51) Int. Cl. A61K 31/445; C07D 401/12; A61K 31/454; C07D 405/12; A61K 31/453; C07D 207/04; A61P 15/00; A61K 31/402; A61K 31/4025; C07D 211/20; A61K 31/4535; C07D 409/12

(52) U.S. Cl. 514/319; 546/201; 514/323; 546/196; 514/320; 546/237; 514/324; 546/202; 548/525; 514/422; 548/576; 514/428

(57) ABSTRACT

The present invention relates to a selective estrogen receptor modulator selected from the group consisting of: (I) or a pharmaceutical salt thereof; useful, e.g., for treating endometriosis and uterine leiomyoma.

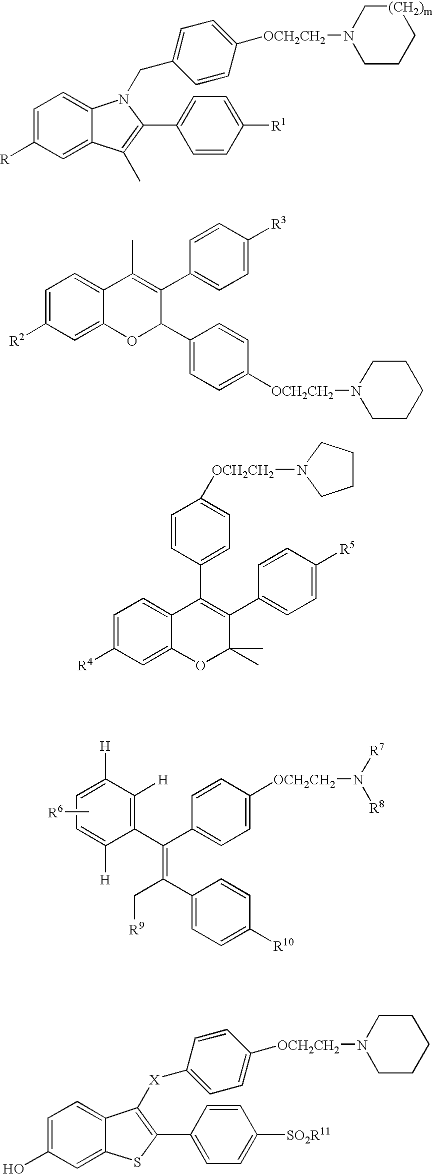

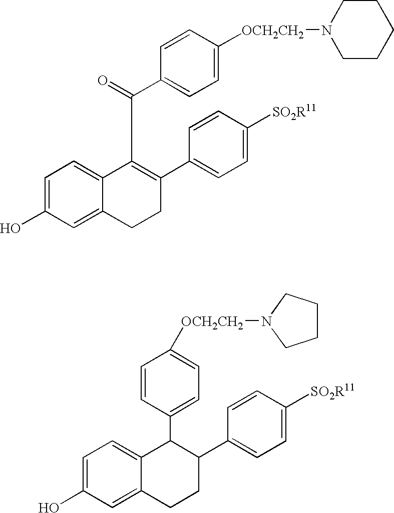

### FIELD OF INVENTION

The present invention is in the field of medicine, particularly in the treatment of gynecological disorders. More specifically, the present invention relates to selective estrogen receptor modulators useful to treat endometriosis and uterine leiomyoma.

### BACKGROUND OF THE INVENTION

Uterine leiomyoma/leiomyomata (uterine fibroid disease) is an old and ever present clinical problem that goes under a variety of names, including uterine fibrosis, uterine hypertrophy, uterine lieomyomata, myometrial hypertrophy, fibrosis uteri, and fibrotic metritis. Essentially, uterine fibrosis is a condition where there is an inappropriate deposition of fibroid tissue on the wall of the uterus. This condition is a cause of dysmenorrhea and infertility in women.

Endometriosis is a condition of severe dysmenorrhea, which is accompanied by severe pain, bleeding into the endometrial masses or peritoneal cavity and often leads to infertility. The symptom's cause appears to be ectopic endometrial growths that respond inappropriately to normal hormonal control and are located in inappropriate tissues. Because of the inappropriate locations for endometrial growth, the tissue seems to initiate local inflammatory-like responses causing macrophage infiltration and a cascade of events leading to initiation of the painful response. Evidence suggests that a cause of uterine fibrosis and endometriosis is an inappropriate response of fibroid tissue and/or endometrial tissue to estrogen.

Many publications have appeared within the last ten years disclosing novel selective estrogen receptor modulators (SERMs). Many of these SERMs, generally speaking, have been found to have a beneficial estrogen agonist activity in the bone and cardiovascular systems with a concomitant beneficial estrogen antagonist activity in the breast. A small, particularly useful subset of such compounds has also been found to have an estrogen antagonist effect in the uterus. A compound with this particularly useful SERM profile holds particular promise in treating uterine leiomyoma/leiomyomata and/or endometriosis.

However, the actual use of these SERM compounds, particularly in pre-menopausal women, has been hampered due to said compound's stimulatory effect on the ovaries. A great need currently exists, therefore, for new SERM compounds that behave as estrogen antagonists in the uterus that do not stimulate the ovaries.

### SUMMARY OF INVENTION

The present invention relates to a sulfonylated SERM compound selected from the group consisting of:

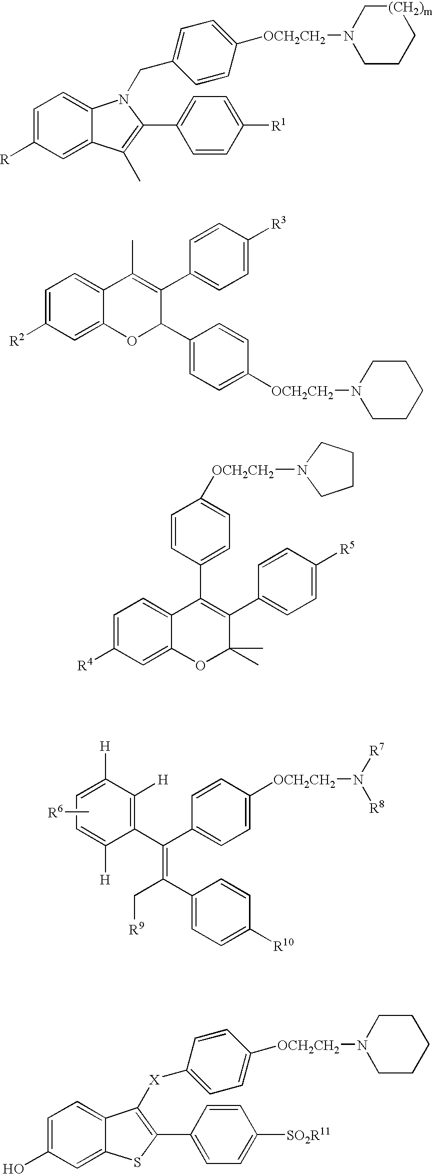

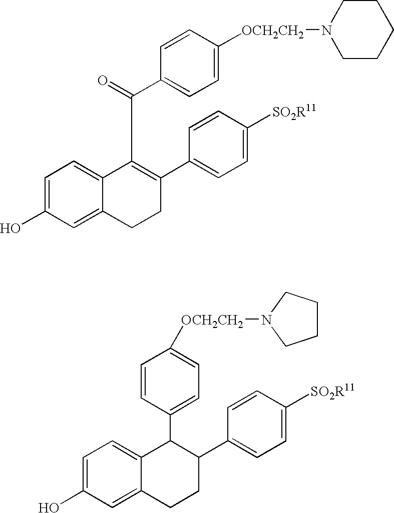

wherein:
m is 1 or 2;
R and R¹ are OH or SO₂R¹¹ provided that one and only one of R or R¹ must be and is SO₂R¹¹;
R² and R³ are OH, OCOC(CH₃)₃ or SO₂R¹¹ provided that one and only one of R² or R³ must be and is SO₂R¹¹;
R⁴ and R⁵ are OH, OCH₃ or SO₂R¹¹ provided that one and only one of R⁴ or R⁵ must be and is SO₂R¹¹;
R⁶ is H, OH, OPO(OH)₂, I or SO₂R¹¹ and R¹⁰ is H, CH(CH₃)₂ or SO₂R¹¹ provided that one and only one of R⁶ or R¹⁰ must be and is SO₂R¹¹;
R⁷ and R⁸ are both methyl or combine with the nitrogen to which they are attached to form a pyrollidinyl ring;
R⁹ is CH₃ or CH₂Cl;
R¹¹ is C₁-C₆ alkyl, C₁-C₆ alkoxy, NR¹²R¹³, CF₃ or CH₂CF₃;
X is CO or O;
R¹² is C₁-C₆ alkyl or phenyl; and
R¹³ is H, C₁-C₆ alkyl or phenyl; or a pharmaceutical salt thereof.

The present invention also relates to a pharmaceutical composition that comprises a compound of the present invention, or a pharmaceutical acid addition salt thereof, and a pharmaceutical carrier. In another embodiment, the pharmaceutical composition of the present invention may be adapted for use in treating endometriosis and/or uterine leiomyoma.

The present invention also relates to methods for treating endometriosis and/or uterine leiomyoma employing a compound of the present invention, or a pharmaceutical acid addition salt thereof.

In addition, the present invention relates to a compound of the present invention, or a pharmaceutical acid addition salt thereof, for use in treating endometriosis and/or uterine leiomyoma. The present invention is further related to the use of a compound of the present invention, or a pharmaceutical acid addition salt thereof, for the manufacture of a medicament for treating endometriosis and/or uterine leiomyoma.

### DETAILED DESCRIPTION

Unless specified otherwise, reference hereafter to a “compound of the present invention” includes the pharmaceutical salts thereof. Certain compounds of the present invention contain an acidic proton, i.e., when R⁶ is OPO(OH)₂. Therefore, the pharmaceutical salts of the present invention include base addition and acid addition salts thereof.

The compounds of the present invention may have one or more chiral centers and may exist in a variety of stereoisomeric configurations. As a consequence of these chiral centers, these compounds of the present invention occur as racemates, mixtures of enantiomers and as individual enantiomers, as well as diastereomers and mixtures of diastereomers. All such racemates, enantiomers, and diastereomers are within the scope of the present invention.

For the purposes of the present invention, as disclosed and claimed herein, the following terms are defined below.

The term “C₁-C₆ alkyl” represents a straight, branched or cyclic hydrocarbon moiety having from one to six carbon atoms, e.g., methyl, ethyl, n-propyl, isopropyl, cyclopropyl, n-butyl, isobutyl, sec-butyl, t-butyl, cyclobutyl, pentyl, cyclopentyl, hexyl, cyclohexyl and the like. Moieties such as a cyclobutylmethylene are also included within the scope of a C₁-C₆ alkyl group. The term “C₁-C₄ alkyl” refers specifically to methyl, ethyl, n-propyl, isopropyl, cyclopropyl, cyclopropylmethyl, n-butyl, isobutyl, sec-butyl, t-butyl and cyclobutyl. A “C₁-C₆ alkoxy” group is a C₁-C₆ alkyl moiety connected through an oxy linkage.

The term “pharmaceutical” when used herein as an adjective means substantially non-deleterious.

A pharmaceutical “acid addition salt” is a salt formed by reaction of the free base form of a compound of the present invention with a pharmaceutical acid, such as described in the Encyclopedia of Pharmaceutical Technology, editors James Swarbrick and James C. Boylan, Vol 13, 1996 “Preservation of Pharmaceutical Products to Salt Forms of Drugs and Absorption”. Specific salt forms include, but are not limited to the: acetate, benzoate, benzenesulfonate, 4-chlorobenzenesulfonate; citrate; ethanesulfonate; fumarate; d-gluconate; d-glucuronate; glutarate; glycolate; hippurate; hydrochloride; 2-hydroxyethanesulfonate; dl-lactate; maleate; d-malate; 1-malate; malonate; d-mandelate; 1-mandelate; methanesulfonate; 1,5 napthalenedisulfonate; 2-naphthalenesulfonate; phosphate; salicylate; succinate; sulfate; d-tartrate; 1-tartrate; and p-toluenesulfonate.

A pharmaceutical “base addition” salt is a salt formed by reaction of the free base form of a compound of formula I with a pharmaceutical base, such as described in the Encyclopedia of Pharmaceutical Technology, editors James Swarbrick and James C.

Boylan, Vol 13, 1996 “Preservation of Pharmaceutical Products to Salt Forms of Drugs and Absorption”. Specific salt form's include, but are not limited to the: calcium, diethanolamine, diethylamine, ethylenediamine, lysine, magnesium, piperazine, potassium, sodium and tromethamine (Tris, Trizma) salts.

The term “patient” as used herein refers to female humans and non-human female animals such as companion animals (dogs, cats, horses and the like).

The terms “treating” and “treat” as used herein, means alleviating, ameliorating, preventing, prohibiting, restraining, slowing, stopping, or reversing the progression or severity of a pathological condition, or sequela thereof, described herein. The term “preventing” means reducing the likelihood that the recipient of a compound of the present invention will incur, further incur or develop any of the pathological conditions, or sequela thereof, described herein.

The term “patient in need thereof” is a patient either suffering from the claimed pathological condition or sequela thereof, or is a patient at a recognized risk thereof, as determined by medical diagnosis, i.e., as determined by the attending physician.

As used herein, the term “effective amount” means an amount of a compound of the present invention that is capable of treating the conditions described herein.

### PREFERRED COMPOUNDS AND EMBODIMENTS OF THE INVENTION

Certain compounds of the invention are particularly interesting and are preferred. The following listing sets out several groups of preferred compounds. It will be understood that each of the listings may be combined with other listings to create additional groups of preferred compounds.
a) R is OH and R¹ is SO₂R¹¹;
b) R² is OH or OCOC(CH₃)₃ and R³ is SO₂R¹¹;
c) R³ is OH or OCOC(CH₃)₃ and R² is SO₂R¹¹;
d) R⁴ is OH or OCOC(CH₃)₃ and R⁵ is SO₂R¹¹;
e) R⁵ is OH or OCOC(CH₃)₃ and R⁴ is SO₂R¹¹;
f) R⁶ is H;
g) R⁶ is OH;
h) R⁶ is OH and is at the para-position;
i) R⁶ is OH and is at the meta-position;
j) R⁶ is OPO(OH)₂ and is at the para-position;
k) R⁶ is I and is at the para-position;
l) R⁶ is SO₂R¹¹ and is at the para-position;
m) R⁶ is SO₂R¹¹ and is at the meta-position
n) R⁷ and R⁸ are both methyl;
o) R⁷ and R⁸ combine with the nitrogen to which they are attached to form a pyrollidinyl ring;
p) R⁹ is CH₃;
q) R⁹ is CH₂Cl;
r) R¹⁰ is H;
s) R¹⁰ is CH(CH₃)₂;
t) R¹⁰ is SO₂R¹¹;
u) R¹¹ is C₁-C₄ alkyl, NR¹²R¹³ or CF₃ and R¹² is C₁-C₄ alkyl and R¹³ is H or C₁-C₄ alkyl;
v) R¹¹ is methyl, ethyl, cyclopropyl, NHCH₃, N(CH₃)₂ or CF₃;
w) R¹¹ is methyl or N(CH₃)₂;
x) R¹¹ is methyl;
y) R¹¹ is N(CH₃)₂;
z) X is CO;
aa) X is O.

The preferred patient of treatment is a female human.

A compound of the present invention is preferably formulated in a dosage unit form, i.e., in an individual delivery vehicle, for example, a tablet or capsule, prior to administration to the recipient woman.

A compound of the present invention is preferably administered orally.

### Synthesis

The compounds of the present invention may be prepared from the corresponding free hydroxy compounds (compounds where a hydroxy moiety is present in place of the required sulfonyl moiety). Said free hydroxy compounds are known in the art and, therefore, are readily accessible. The free hydroxy compound may be converted to a compound of the present invention, for example, by converting said hydroxy moiety to an aryl triflate employing typical procedures for such a transformation. Once prepared, the triflate may be coupled with a desired thiolate (compound of the formula HSR¹¹); via palladium catalyzed cross coupling (see, e.g., Zheng, et al., J. Org. Chem., 63: 9606-9607, 1998.). Once coupled, the compound may be oxidized or otherwise manipulated employing standard organic synthesis methodology to form the compounds of the present invention.

### Formulation

Because the free base form of a compound of the present invention contains a basic moiety (i.e., amino), said compound may be formulated as a pharmaceutical acid addition salt, e.g., as the hydrochloride salt or as a salt described in “Handbook of Pharmaceutical Salts Properties, Selection and Use”, Weinheim, N.Y.: VHCA; Wiley-VCH, 2002.

The present pharmaceutical compositions are prepared by known procedures using well-known and readily available ingredients. In making the formulations of the present invention, the active ingredient (a compound of the present invention) will usually be mixed with a carrier, or diluted by a carrier, or enclosed within a carrier which may be in the form of a capsule, sachet, paper or other container. When the carrier serves as a diluent, it may be a solid, semisolid or liquid material which acts as a vehicle, excipient or medium for the active ingredient.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents.

### Biological Assays

Estrogen Receptor Binding Assay: Representative compounds of the present invention are screened for binding affinity to both estrogen receptor types (ERα and ERβ). This competition binding assay measures the compound's ability to displace ³H-estradiol and generates IC₅₀ and Kᵢ values for both receptor types.

This competition binding assay is run in a buffer containing 50 mM Hepes, pH 7.5, 1.5 mM EDTA, 150 mM NaCl, 10% glycerol, 1 mg/mL ovalbumin and 5 mM DTT, using 0.025 μCi per well ³H-Estradiol (NEN #N-ET517 at 118 Ci/mmol, 1 mCi/mL), 10 ng/well ERAlpha or ERbeta receptor (PanVera). A compound of the present invention is added at 10 different concentrations. Non-specific binding is determined in the presence of 1M of 17-B Estradiol. The binding reaction (140 μl) is incubated for 4 hours at room temperature, then 70 μl of cold DCC buffer is added to each reaction (DCC buffer contains per 50 mL of assay buffer, 750 mg of charcoal (Sigma) and 250 mg of dextran (Pharmacia)). Plates are mixed 8 minutes on an orbital shaker at 4° C. Plates are then centrifuged at 3,000 rpm at 4° C. for 10 minutes. An aliquot of 120 μl of the mix is transferred to another 96-well, white flat bottom plate (Costar) and 175 μl of Wallac Optiphase “Hisafe 3” scintillation fluid is added to each well. Plates are sealed and shaken vigorously on an orbital shaker. After an incubation of 2.5 hours, the plates are read in a Wallac Microbeta counter. The data is used to calculate an IC₅₀ and % Inhibition at 10 μM. The K_{d} for ³H-Estradiol is determined by saturation binding to ER alpha and ER beta receptors. The IC₅₀ values for test compounds are converted to Kᵢ using Cheng-Prusoff equation and the K_{d} determined by saturation binding assay.

Ishikawa Cell Proliferation Assay: This assay measures cell proliferation (using an alkaline phosphatase readout) in both an agonist mode in the presence of a compound of the present invention alone, and in an antagonist mode in which the ability of a compound of the present invention to block estradiol stimulation of growth is measured.

Ishikawa human endometrial tumor cells are maintained in MEM (minimum essential medium, with Earle's salts and L-Glutamine, Gibco BRL, Gaithersburg, Md.), supplemented with 10% fetal bovine serum (FBS) (V/V), (Gibco BRL). One day prior to assay, growth media is changed to assay medium, DMEM/F-12 (3:1) (Dulbecco's Modified Eagle Medium: Nutrient Mixture F-12, 3:1 Mixture, phenol red-free, Gibco BRL) supplemented with 5% dextran coated charcoal stripped fetal bovine serum (DCC-FBS) (Hyclone, Logen, Utah), L-Glutamine (2 mM), MEM sodium pyruvate (1 mM), HEPES (N-[2-hydroxyethyl]piperazine-N′-[2-ethanesulfonic acid]2 mM) all from Gibco BRL). After an overnight incubation, Ishikawa cells are rinsed with Dulbecco's Phosphate Buffered Saline (1×) (D-PBS) without Ca⁺² and Mg⁺² (Gibco BRL), and trypsinized by a 3 minute incubation with 0.25% Trypsin/EDTA, phenol red-free (Gibco BRL). Cells are resuspended in assay medium and adjusted to 250,000 cells/mL. Approximately 25,000 cells in a 100 μl media are added to flat-bottom 96 wells microculture plates (Costar 3596) and incubated at 37° C. in a 5% CO₂ humidified incubator for 24 hours. The next day, serial dilutions of compounds are prepared in assay medium (at 6 times the final concentration in the assay). The assay is run in dual mode, agonist and antagonist modes.

For the agonist mode, plates receive 25 μl/well of assay medium followed by 25 μl/well of a diluted compound of the present invention (at 6× the final concentrations).

For the antagonist mode, plates receive 25 μl/well of 6 nM E₂ (β-Estradiol, Sigma, St. Louis, Mo.) followed by 25 μl/well of a diluted compound of the present invention (at 6× the final concentrations). After an additional 48-hour incubation at 37° C. in a 5% CO₂ humidified incubator, media is aspirated from wells and 100 μl fresh assay medium is added to each microculture. Serial dilutions of compounds are prepared and added to the cells as described above. After an additional 72 hour incubation at 37° C. in a 5% CO₂ humidified incubator, the assay is quenched by removing media and rinsing plates twice in Dulbecco's Phosphate Buffered Saline (1×) (D-PBS) (Gibco BRL). The plates are dried for 5 minutes and frozen at −70° C. for at least 1 hour. The plates are then removed from the freezer and allowed to thaw at room temperature. To each well, 100 μl of 1-Step™ PNPP (Pierce Chemical Company, Rockford, Ill.) is added. After a 20-minute incubation, plates are read on a spectophotometer at 405 nm.

The data is fitted to a linear interpolation to derive EC₅₀ (for agonist mode) or IC₅₀ (for antagonist mode) values. For the antagonist mode, a % efficacy for each compound is calculated versus E2 (1 nM) alone. For the agonist mode, a % efficacy for each compound is calculated versus the response to tamoxifen.

MCF-7 Proliferations Assay: The MCF-7 cell line is derived from a human breast adenocarcinoma and is used as an indicator of potential antiproliferative activity in breast epithelium.

MCF-7 breast adenocarcinoma cells (ATCC HTB 22) are maintained in MEM (minimal essential medium, phenol red-free, Gibco BRL) supplemented with 10% fetal bovine serum (FBS) (V/V), L-glutamine (2 mM), sodium pyruvate (1 mM), HEPES ((N-[2-hydroxyethyl]piperazine-N′-[2-ethanesulfonic acid]10 mM}, non-essential amino acids (0.1 mM) and Penicillin Streptomycin (1×). Seven days prior to assay, MCF-7 cells are switched to assay media which is the same as maintenance medium except supplemented with 10% dextran-coated charcoal-stripped fetal bovine serum (DCC-FBS) assay medium in place of 10% FBS. MCF-7 cells are removed from flasks using 10× Trypsin EDTA (phenol red free, Gibco BRL) and diluted to 1× in (Ca++/Mg++ free HBSS (phenol red-free). Cells are adjusted to 80,000 cells/mL in assay medium. Approximately 8,000 cells (100 μl) are added to each well in 96 well Cytostar T scintillation plates (Amersham) and incubated at 37° C. in a 5% CO₂ humidified incubator for 24 hours to allow cell adherence and equilibration after transfer. Serial dilutions of a compound of the present invention are prepared in assay medium at 4× the final desired concentration). A 50 μl aliquot of test compound dilutions (at 4× the final assay concentration) is transferred to duplicate wells followed by 50 μl assay medium for the agonist mode or 50 μl of 40 μM of E2 for the antagonist mode to a final volume of 200 μl. For each of the agonist plates, a basal level (media) and a maximum stimulated level (with 1 μM E2) is determined. For each of the antagonist plates, a basal level (media) and an E2 (10 pM) alone control is determined. After an additional 48 hours at 37° C. in a 5% CO₂ humidified incubator, 20 μl of assay medium containing 0.01 μCi of ¹⁴C-thymidine (52 mCi/mmol, 50 μCi/ul, Amersham) is added to each well. The plates are incubated overnight in the same incubator and then counted on the Wallac Microbeta counter. The data is averaged to calculate an IC₅₀ and % inhibition @ 1 μM for the antagonist mode. For the agonist mode, an EC₅₀ and percent of maximum E2 stimulation and concentration of maximum stimulation is calculated.

3-Day Rat Uterus Antagonist Assay: This model for uterine antagonism utilizes immature (3 week old) female rats that are highly sensitive to estrogenic stimulation of the uterus given that their circulating estrogen levels are prepubertal. The uteri from immature rats are fully responsive to exogenous estrogen, yet are quiescent in the absence of exogenous estrogen. Administration of exogenous estrogen to immature rats produces a reliable elevation of uterine weight, which can be used to study uterine antagonist effects. The rats are treated with both estradiol and 4 different concentrations of a compound of the present invention for 3 days and then uterine wet weights are measured.

Nineteen to twenty-one day old (or 45-50 g) female rats are orally treated with E2 (0.1 mg/kg, a maximal stimulatory estrogenic stimulus for reliably increasing uterine weight) and 10, 1.0, 0.1 and 0.01 mg/kg test compound for 3 days, 6 rats per group. Test compounds are dissolved in 20% β-hydroxycyclodextrin and administered by oral gavage in a volume of 0.2 mL daily (15 min. prior to the ethynyl estradiol gavage). A vehicle control, E2 alone and E2+raloxifene are also done as controls. The animals are fasted overnight following the final dose. On the following morning, the animals are weighed, then euthanized (by carbon dioxide asphyxiation) and the uteri rapidly collected (via a mid-line ventral incision) and weighed.

Uterine weight/body weight ratios (UWR) are calculated for each animal. The percent inhibition of the estrogen-induced response is then calculated by the following formula: percent inhibition=100×(UWRestrogen−UWRtest compound/UWRestrogen−UwRcontrol). ED₅₀ values are derived from a semi-log regression analysis of the linear aspect of the dose response curve. Both the UWR data and the percent inhibition data are statistically analyzed by one way analysis of variance (ANOVA) with post-hoc testing by Fisher's PLSD when indicated by a p≦0.05. Statistical analyses are performed using the Statview® 4.0 software package.

4-Day OVX Rat Uterine Agonist Assay: In order to assure that a test compound does not have any partial uterine agonist activity, compounds are administered to mature, ovariectomized rats.

Seventy-five day old rats are ovariectomized and treatment is started 14 days later when circulating estradiol levels have reached minimal levels. After 4 days of treatment with 3 doses of a compound of the present invention, (6 rats per group) body weight, uterine wet weight and uterine eosinophil peroxidase (EPO) activity are measured. Cholesterol levels are also measured to compare relative ability to lower cholesterol with other SERMs. If there is any question of uterine stimulation, histological examination will determine epithelial cell height.

10-Day Rat Hormone (Ovarian Stimulation) Screen: An initial, first screen for ovarian toxicity is conducted using a 10-day rat hormone study to measure estradiol and luteinizing hormone levels after compound administration. This screen is conducted by administering compound by oral gavage for 10 days to mature (9-10 week old) F344 female rats. Trunk blood is collected by rapid decapitation for evaluation of LH and estradiol levels approximately 2 hours after the 10^{th} dose. Serum, obtained by centrifugation, is removed and stored frozen below −60° C. until assayed. Serum levels of LH and estradiol are measured using radioimmunoassay (RIA) methods.

Rat LH primary antibody and reference preparations (rat LH:RP-3) are obtained from Dr. A. F. Parlow, Director, Pituitary Hormones and Antisera Center, Harbor-UCLA Medical Center, Torrance, Calif. The LH assay upper limits of detection are 30 ng/mL and the lower limits of detection are 0.1 ng/mL for the 100 μl samples.

E2 Clinical Assays. DiaSorin s.r.l., Saluggia (Vercelli), Italy. The upper limit of detection is 1000 pg/mL and the lower limit of detection is 5 pg/mL.

35-Day Ovary-Intact Rat Bone Assay: While previous SERMs, including raloxifene have shown efficacy in preventing bone loss in OVX rats, the possibility of interference with estrogen-regulated turnover in ovary-intact rats needs to be addressed.

This assay is done in mature rats with concentrations based on the demonstrated efficacy in the 3-day assay. Generally, at least three concentrations are chosen based on multiples of the ED₅₀ generated therein. These multiples are generally 1×, 10× and 30× the ED₅₀. A compound of the present invention is administered to an OVX rat for 35 days and is compared to control, ovariectomized, and/or GnRH-administered rats. Femurs, tibiae, uteri, ovaries and serum are taken for further analyses. DEXA (Dual Energy X-ray Absorptivity), CT (Computed Tomography) and histologic analysis are done on the long bones to assess any changes. CT scans of the distal femur are done to calculate BMD (bone mineral density), cross sectional area and BMC (bone mineral content). Bone strength measurements (load to failure) may also be done to determine consequences of any bone mass or material changes. Uterine and ovarian histology are examined to confirm long term dosing effects of uterine efficacy and potential ovarian stimulation. The serum is analyzed for LH and E2 levels as a possible indicator of ovarian effects.

### Utilities

The diseases, disorders or conditions for which a compound of the present invention is useful in treating include, but are not limited to, (1) uterine cancer; (2) endometriosis; (3) uterine leiomyoma/leiomyomata; (4) post-menopausal osteoporosis, i.e., osteoporosis caused by the loss of bone that results from a lack of endogenous estrogen such as occurs in a woman following cessation of menstration due to natural, surgical, or other processes; and (5) estrogen receptor positive (ER+) breast cancer, particularly the prevention thereof. Treatment of uterine leiomyoma/leiomyomata as described herein, also contemplates the reduction of the occurrence or severity of the associated symptoms such as pain, urinary frequency, and uterine bleeding.

### Dose

The specific dose administered is determined by the particular circumstances surrounding each situation. These circumstances include, the route of administration, the prior medical history of the recipient, the pathological condition or symptom being treated, the severity of the condition/symptom being treated, and the age of the recipient. The recipient patient's physician should determine the therapeutic dose administered in light of the relevant circumstances.

Generally, an effective minimum daily dose of a compound of the present invention will exceed about 5 mg. Typically, an effective maximum daily dose will not exceed about 350 mg. The exact dose may be determined, in accordance with the standard practice in the medical arts of “dose titrating” the recipient; that is, initially administering a low dose of the compound, and gradually increasing the does until the desired therapeutic effect is observed.

1. A compound selected from the group consisting of: 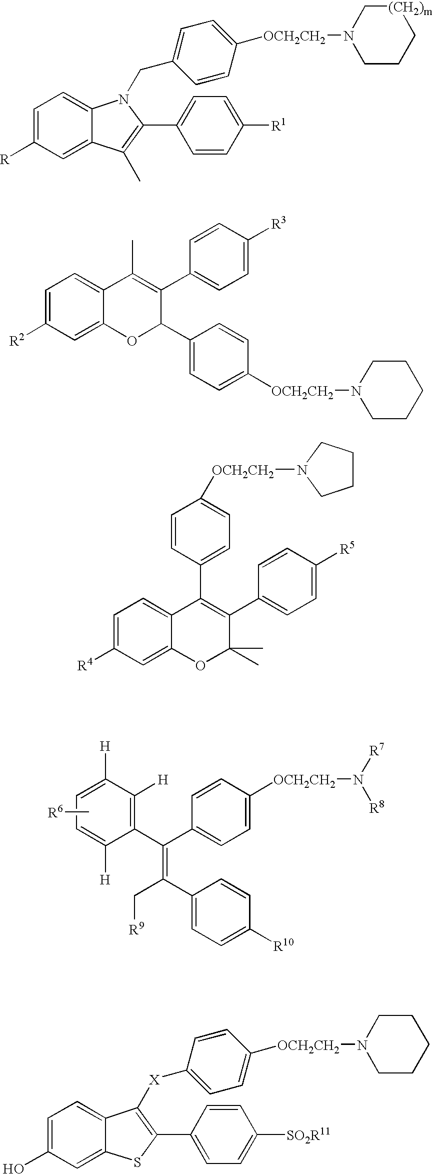 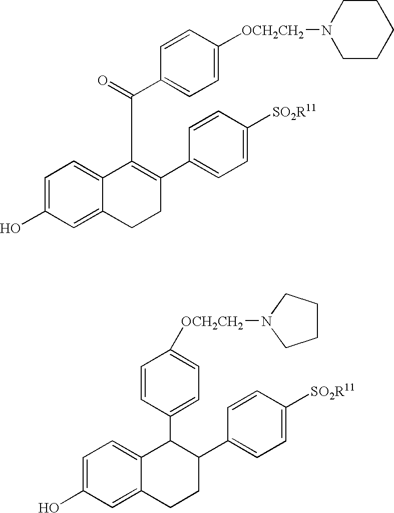
wherein:
m is 1 or 2;
R and R¹ are OH or SO₂R¹¹ provided that one and only one of R or R¹ must be and is SO₂R¹¹;
R² and R³ are OH, OCOC(CH₃)₃ or SO₂R¹¹ provided that one and only one of R² or R³ must be and is SO₂R¹¹;
R⁴ and R⁵ are OH, OCH₃ or SO₂R¹¹ provided that one and only one of R⁴ or R⁵ must be and is SO₂R¹¹;
R⁶ is H, OH, OPO(OH)₂, I or SO₂R¹¹ and R¹⁰ is H, CH(CH₃)₂ or SO₂R¹¹ provided that one and only one of R⁶ or R¹⁰ must be and is SO₂R¹¹;
R⁷ and R⁸ are both methyl or combine with the nitrogen to which they are attached to form a pyrollidinyl ring;
R⁹ is CH₃ or CH₂Cl;
R¹¹ is C₁-C₆ alkyl, C₁-C₆ alkoxy, NR¹²R¹³, CF₃ or CH₂CF₃;
X is CO or O;
R¹² is C₁-C₆ alkyl or phenyl; and
R¹³ is H, C₁-C₆ alkyl or phenyl; or a pharmaceutical salt thereof.

2. The compound of claim 1 selected from the group consisting of: 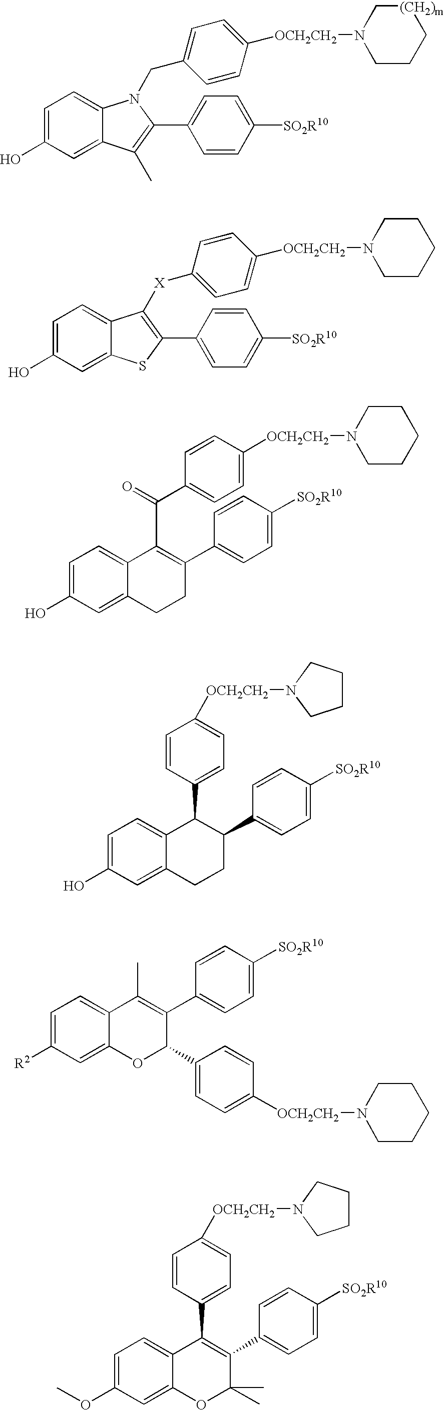 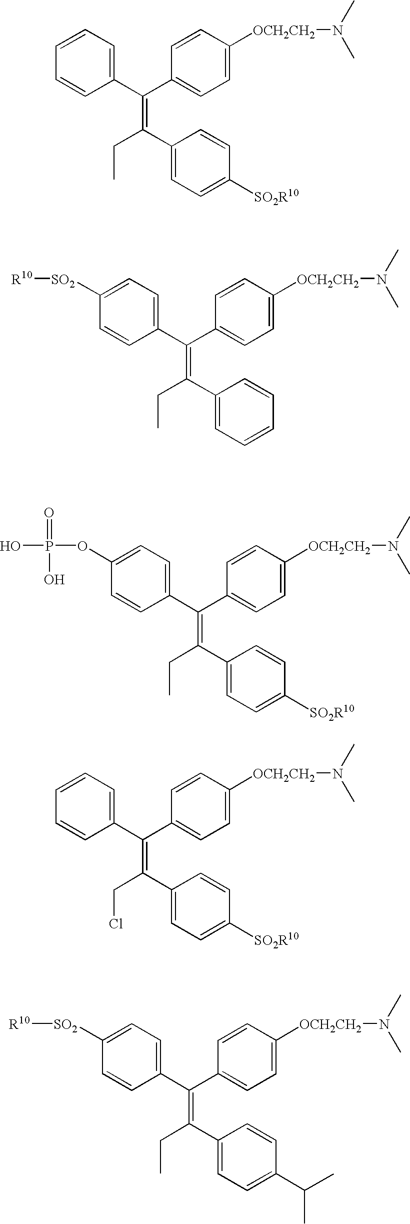 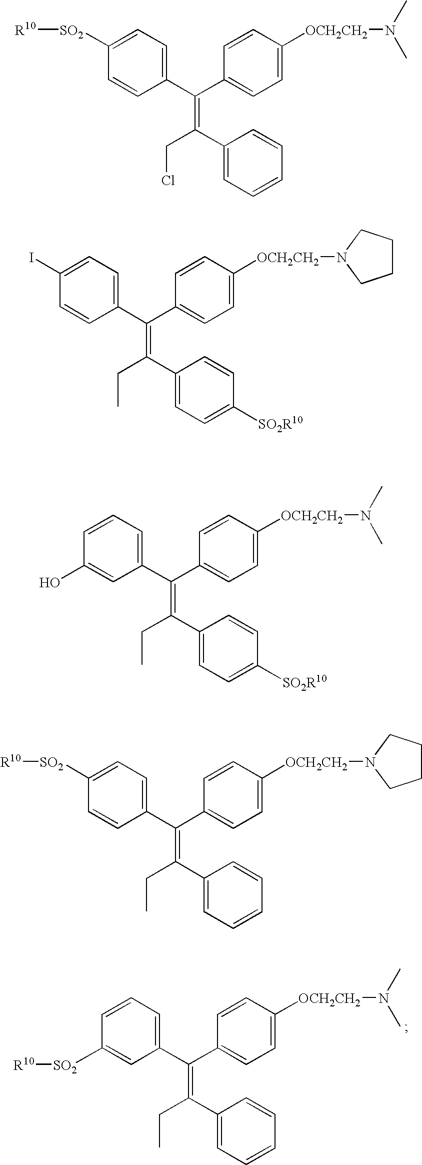
or a pharmaceutical salt thereof.

3. The compound of claim 2 wherein R¹¹ is C₁-C₄ alkyl, CF₃ or NR¹²R¹³ and R¹² is C₁-C₄ alkyl and R¹³ is H or C₁-C₄ alkyl.

4. The compound of claim 2 wherein R¹¹ is methyl, ethyl, cyclopropyl, CF₃, NHCH₃ or N(CH₃)₂.

5. The compound of claim 4 wherein R¹¹ is methyl or N(CH₃)₂.

6. The compound of claim 5 wherein R¹¹ is methyl.

7. The compound of claim 5 wherein R¹¹ is N(CH₃)₂.

8. (canceled)

9. A method of treating endometriosis comprising administering to a patient in need thereof an effective amount of a compound of claim 1.

10. A method of treating uterine leiomyoma comprising administering to a patient in need thereof an effective amount of a compound of claim 1.

11. (canceled)
